(19) United States
(12) Patent Application Publication
(10) Pub. No.: US 2005/0228222 A1
(43) Pub. Date: Oct. 13, 2005

(54) Method of manufacturing endoscope flexible tube

(75) Inventor: Satoshi Furumi, Tokyo (JP)

Correspondence Address: SCULLY SCOTT MURPHY & PRESSER, PC, 400 GARDEN CITY PLAZA, SUITE 300, GARDEN CITY, NY, 11530, US

(73) Assignee: Olympus Corporation, Tokyo (JP)

(21) Appl. No.: 11/100,242

(22) Filed: Apr. 6, 2005

(30) Foreign Application Priority Data
Aug. 11, 2004 (JP) 2004-234586
Apr. 9, 2004 (JP) 2004-115533

(51) Int. Cl. A61B 1/00

(52) U.S. Cl. 600/101

(57) ABSTRACT

The present invention provides a method of manufacturing a flexible tube for an endoscope including heating a flexible tube member formed at least partly of metal and covering an outer coat thereon, wherein the flexible tube member is heated by irradiating a near infrared ray. The near infrared ray can heat metal satisfactorily and selectively in comparison with other materials such as synthetic resin or the like. Therefore, heating of the portion other than the surface of the flexible tube member can be restrained. Therefore, even when synthetic resin is used for a jig, deformation of the jig can be restrained. The preferred wavelength of the near infrared ray is from about 0.8 to about 2.0 μm.

### CROSS-REFERENCE TO RELATED APPLICATION

This application is based upon and claims the benefit of priority from prior Japanese Patent Application Nos. 2004-115533 filed on Apr. 9, 2004 and 2004-234586 filed on Aug. 11, 2004, the entire contents of both of which are incorporated herein by reference.

### BACKGROUND OF THE INVENTION

1. Field of the Invention

The present invention relates to a method of manufacturing an endoscope flexible tube disposed in the endoscope for medical and industrial use.

2. Description of the Related Art

An endoscope flexible tube disclosed in JP-A-11-42204 is formed by covering an outer periphery of a flex, which is a metal band strip wound into a helical shape, with a mesh tube whereof at least a part of an element wire or a bundle of element wires is formed of metal. The endoscope flexible tube is formed by covering the outer peripheral surface of the flexible tube member with an outer coat as a thermoplastic resilient member by extrusion molding. In order to enhance a bonding force between the flexible tube member and the outer coat, the surface of the flexible tube member is heated by a device such as an infrared heater (middle wavelength), a heat gun, a ceramic heater, a far infrared heater, a high-frequency heater, or a hot air circulating oven, or a combination thereof before covering with the outer coat. Accordingly, melting of the outer coat is promoted by the heat of the flexible tube member, and the outer coat is bonded with the flexible tube member. Accordingly, the flexible tube can be manufactured simply without necessity of adhesive agent. In the endoscope flexible tube as such, the bonding force between the mesh tube and the outer coat is strong, separation between the mesh tube and the outer coat hardly occurs, and hence the outer coat hardly gathers into wrinkles, thereby ensuring uniform flexibility of the flexible tube and good followability to torsional deformation, and reducing possibility of kinking.

A key point of disclosure in JP-A-11-42204 is to perform preheating to increase the surface temperature of the flexible tube member (mesh tube) in advance to a temperature higher than a deformation temperature of synthetic resin material used for the outer coat before coating the outer coat in order to obtain strong and stable bonding force between the flexible tube material and the outer coat. Preheating of the flexible tube member which has been introduced hitherto is performed by the infrared heater of middle wavelength, the ceramic heater, the far infrared heater, the high-frequency heater, which are well known.

In a method of manufacturing an endoscope flexible tube disclosed in JP-A-2001-70233, a column shaped core member formed of synthetic resin material or the like having resiliency, elasticity, and heat-resistant property is used instead of a core metal using a metal pipe as a jig used in the manufacturing process. The flexible tube is formed by winding a helical-shaped flex on the core member, covering the outer peripheral surface of the flex with a mesh tube, and covering the mesh tube with an outer coat. Then, the core member is pulled out. The length of the core member extends and the outer diameter of the core member reduces to a value smaller than the inner diameter of the flex because of this pulling. Then, the core member is pulled out from the flexible tube including the flex, the mesh tube, and the outer coat. Therefore, when pulling the core member from inside the flex, the flex is prevented from deforming that would be caused by the friction between the core member and the flex if the diameter of the core member did not became small.

When manufacturing the endoscope flexible tube by applying a technology disclosed in JP-A-11-42204 to a technology using the core member of synthetic resin material disclosed in JP-A-2001-70233, the core member may be deformed by heating of the flexible tube member. It is because when heating the surface of the flexible tube member, the core member of synthetic resin used as a jig absorbs energy generated when the surface of the flexible tube member is heated simultaneously with the flexible tube member.

### BRIEF SUMMARY OF THE INVENTION

In the present invention, when manufacturing the flexible tube for an endoscope by heating a flexible tube member (flexible tube before covered by an outer coat) including at least metal before covering the flexible tube member with the outer coat, heating of the flexible tube member is performed by utilizing a near infrared ray. As described later, by heating the flexible tube member by the near infrared ray, heating to a desired temperature is achieved in a shorter time than the case in which the flexible tube member is heated by an infrared ray of middle wavelength or the case in which the flexible tube member is placed in the atmosphere furnace for heating. Therefore, the time required for manufacturing the flexible tube can be shortened.

When heating by the near infrared ray, the heat absorption coefficient of metal is higher than the heat absorption coefficient of synthetic resin. Therefore, when the near infrared ray is used to heat the flexible tube member including metal in a state in which a core member including the synthetic resin material is contained therein, a rapid increase in temperature of the flexible tube member is achieved while controlling an increase in temperature of the core member to a low degree. Therefore, even when the flexible tube member reaches a temperature which is sufficiently high to bond the outer coat, deformation of the core member due to temperature increase can be prevented.

The peak of strength of the near infrared ray is preferably from 0.8 μm to 2.0 μm.

Heating by the near infrared ray increases the temperature of the surface of the flexible tube member that is to come into contact with the outer coat to a high temperature. Thus, the heat originated from the near infrared ray melts and deforms the outer coat so as to promote bonding between the flexible tube member and the outer coat. Therefore, the near infrared ray is preferably irradiated from the outside of the flexible tube member, because it is suitable to heat the outer surface of the flexible tube member.

The flexible tube member is preferably provided with a mesh tube including an element wire (or a bundle of element wires) which are at least partly formed of metallic material weaved therein outside the flex formed of metal band strip wound into a helical shape.

In this case, the mesh tube preferably contains at least one of stainless alloy, copper, brass, tungsten, and iron. More specifically, the mesh tube is preferably formed of stainless-steel.

The mesh tube may contain non-metallic material in addition to metallic material. Preferable non-metallic material includes synthetic resin, silk string, and kite string.

The synthetic resin for the core member is preferably silicone rubber.

A material for the outer coat to cover the flexible tube member may be thermoplastic polyurethane (TPU), polypropylene (PP), polyethylene-terephthalate (PET), soft vinyl-chloride, polyolefin, polyester, polyethylene, or a composite thereof.

The outer coat is preferably formed with a coating layer of a higher melting temperature than that of the outer coat in order to improve heat-resistant property or chemical-resistant property.

The method of coating the flexible tube member with the outer coat includes extrusion molding and dipping. It is also possible to fit the outer coat formed into tubular shape in advance on the flexible tube member.

### BRIEF DESCRIPTION OF THE SEVERAL VIEWS OF THE DRAWINGS

These and other features, aspects, and advantages of the apparatus and methods of the present invention will become better understood with regard to the following description, appended claims, and accompanying drawings where:

FIG. 1 is a perspective view showing a general configuration of an endoscope according to a first embodiment;

FIG. 2A and FIG. 2B show the structure of a flexible tube for the endoscope according to the first embodiment, in which FIG. 2A is a schematic drawing of the flexible tube, and FIG. 2B is a schematic cross-sectional view of the flexible tube showing a state in which a core member is disposed within the flexible tube;

FIG. 3 is a schematic drawing showing a state in which the outer periphery of the flexible tube member of the endoscope is covered with the outer coat according to the first embodiment;

FIG. 4 is a graph showing heat absorption coefficients of light irradiated to metallic material and synthetic resin material used for the flexible tube for the endoscope with respect to the wavelength of the light according to the first embodiment;

FIG. 5 is a graph showing surface temperatures of the metallic material and the synthetic resin material with respect to time period during which the light with wavelength from 0.8 μm to 2.0 μm is irradiated to the metallic material and the synthetic resin material used for the flexible tube for the endoscope according to the first embodiment;

FIG. 6 is a graph showing a surface temperature of the flexible tube member with respect to the heating time during which the flexible tube member is disposed and heated in an atmosphere furnace at 450° C. after having irradiated light with wavelength within the range of the near infrared ray and light with wavelength within the range of the infrared ray on the core member used for manufacturing the flexible tube for the endoscope according to the first embodiment; and

FIG. 7 is a schematic drawing showing a state in which an outer periphery of a flexible tube member of an endoscope is covered with an outer coat according to a second embodiment.

### DETAILED DESCRIPTION OF THE INVENTION

Embodiments of the invention will be described below with reference to the accompanying drawings.

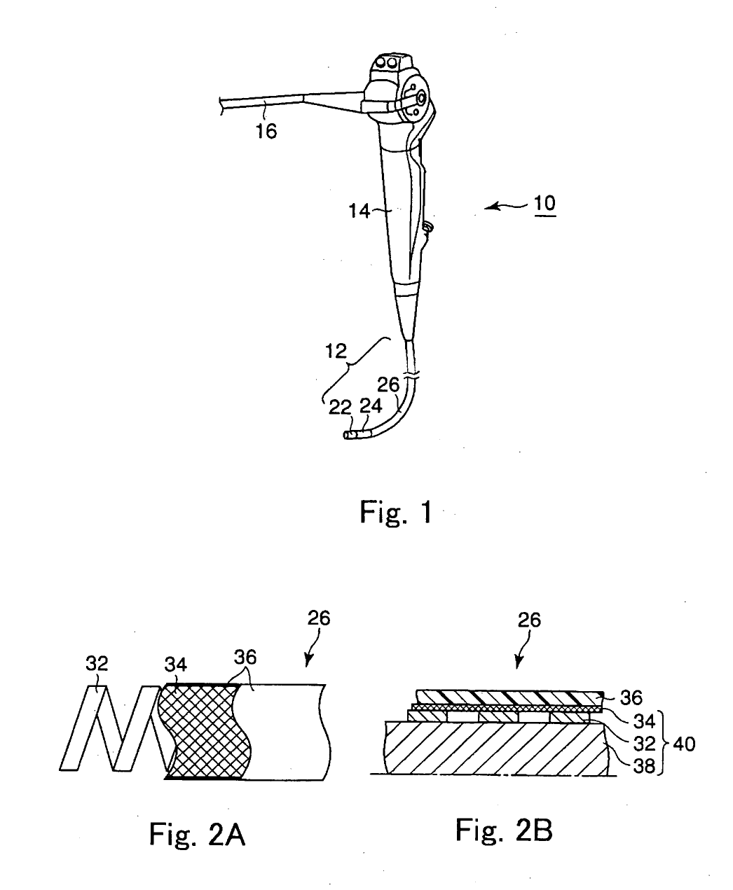

Referring now to FIG. 1 to FIG. 6, a first embodiment will be described.

As shown in FIG. 1, for example, an endoscope 10 for medical use includes an insertion portion 12 which is elongated and has flexibility, a final operating element 14 provided at the proximal end of the insertion portion 12, and a universal cord 16 extending from the final operating element 14.

The insertion portion 12 includes a hard distal portion 22, a bending portion 24 which is connected to the distal portion 22 and is bendable, and a flexible tube 26 connected to the proximal end of the bending portion 24 at one end and connected at the proximal end to the final operating element 14 at another end.

As shown in FIG. 2A, the flexible tube 26 includes a flex 32, a mesh tube 34 disposed on the outer periphery of the flex 32, and an outer coat 36 covering the outer periphery of the mesh tube 34. The flex 32 is formed by winding a metal band strip into a helical shape. The mesh tube 34 includes, for example, an element wire or a bundle of element wires formed of metallic material weaved therein. The element wire or the bundle of element wires of the mesh tube 34 may be formed at least partly of metallic material. For example, the element wire may be configured in such a manner that the outer periphery of non-metallic material is covered with metallic material. Therefore, the element wire formed of metallic material such as stainless steel alloy, copper, brass, tungsten, and iron, or synthetic resin, silk string, kite string or the like covered and combined on the outer periphery thereof with non-metallic material selected from synthetic resin, silk string, kite string and the like are used as needed. In this specification, a case in which the mesh tube 34 is formed of stainless steel material is described.

The outer periphery of the mesh tube 34 is covered with the outer coat 36 formed of thermoplastic resilient member by extrusion molding or dipping. The thermoplastic resilient member may be formed of, for example, thermoplastic polyurethane (TPU), polypropylene (PP), polyethylene terephthalate (PET), soft vinyl-chloride, polyolefin, polyester, polyethylene, or a composite thereof.

Although not shown in the drawings, a coating layer is preferably formed on the outer peripheral surface of the outer coat 36 in order to improve its heat-resistant property or chemical-resistant property of the outer coat 36. The melting temperature of the coating layer is set to a value higher than that of the outer coat 36.

Subsequently, a process of manufacturing the flexible tube 26 configured as described above will be described.

First, a core member 38 having a longitudinal length longer than that of the flexible tube 26 to be manufactured (see FIG. 2B) is prepared. The core member 38 is formed into a column shape or into a cylindrical shape of synthetic resin having resiliency, elasticity, and heat-resistant property. The synthetic resin material is, for example, silicone rubber. Therefore, the core member 38 has such property that the outer diameter thereof is reduced when it is pulled from both ends (pulled longitudinally in opposite directions), and restored to its original outer diameter when released. The original outer diameter of the core member 38 is the same as the inner diameter of the flex 32. The outer periphery of the core member 38 is preferably applied with anti-friction agent for reducing friction drag with respect to the inner peripheral surface of the flex 32.

The flex 32 is tightly wound on the outer periphery of the core member 38 (see FIG. 2B). The outer peripheral surface of the flex 32 is preferably applied with mold lubricant (anti-friction agent) for reducing friction drag with respect to the inner peripheral surface of the mesh tube 34.

The mesh tube 34 is disposed on the outer periphery of the flex 32 (see FIG. 2B). In this manner, as shown in FIG. 2B, a flexible tube member 40 is configured by the core member 38, the flex 32, and the mesh tube 34.

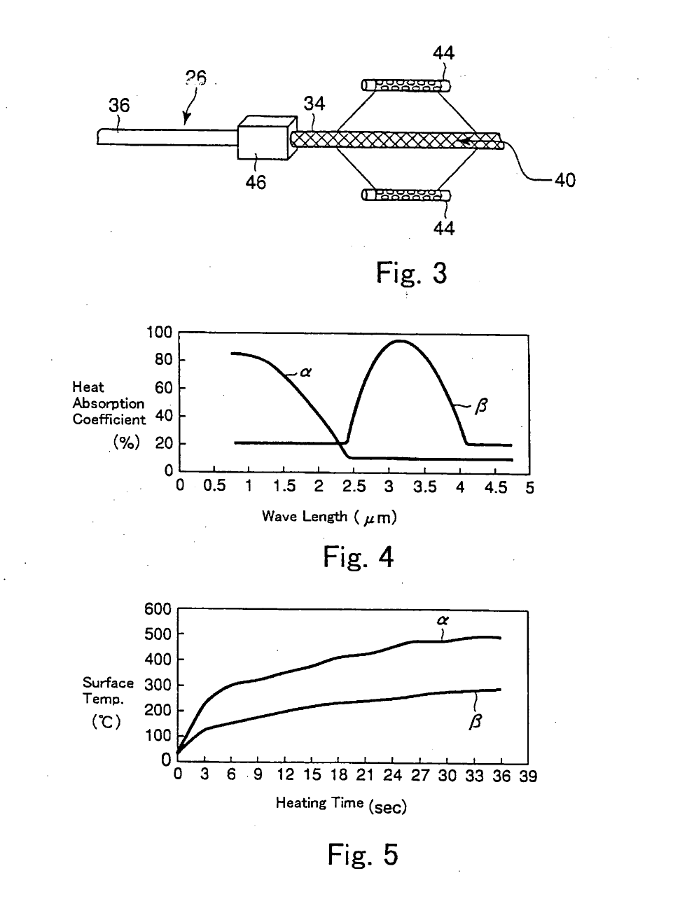

As shown in FIG. 3, the flexible tube member 40 is heated by an infrared heater 44 from the outside. The infrared heater 44 includes one or more (many) light-emitting members (not shown) that emit light having a wavelength in the range of the near infrared ray. The light-emitting member is caused to emit light and irradiates the outer surface of the flexible tube member 40 entirely and evenly to heat the flexible tube member 40. At this time, the temperature of the outer peripheral surface of the flexible tube member 40, that is, of the outer peripheral surface of the mesh tube 34, is increased to at least a softening temperature of the outer coat 36. The wavelength of the near infrared rays emitted from the respective light-emitting members at the moment when the maximum value of emission spectrum is obtained resides in the range, for example, from about 0.8 μm to about 2.0 μm.

After having increased the temperature of the outer periphery of the flexible tube member 40 to at least the softening temperature of the outer coat 36, the outer peripheral surface of the flexible tube member 40 is immediately covered with the outer coat 36. For example, the flexible tube member 40 is passed through a coating device 46 such as an extrusion molding device or a dipping device. Then, since the outer peripheral surface of the flexible tube member 40 is coated with the outer coat 36, the inner peripheral surface of the outer coat 36 is warmed up and is softened by heat from the outer peripheral surface of the flexible tube member 40, the outer coat 36 can be impregnated easily into the clearances (the spaces between the element wires) on the mesh tube 34, and the resin material forming the outer coat 36 gets into the clearances on the mesh tube 34. The flexible tube 26 is cooled by air or the like in this state. At this time, the outer coat 36 gets into the clearances on the mesh tube 34 until the outer coat 36 is decreased in temperature to a hardening temperature. In this manner, the outer coat 36 and the mesh tube 34 are tightly adhered to each other. Therefore, the flexible tube 26 as shown in FIG. 2B is obtained.

By placing the infrared heater 44 for heating the flexible tube member 40 to the coating device 46 of the outer coat 36 such as the extrusion molding device or the dipping device as close as possible, the outer periphery of the flexible tube member 40 can be covered with the outer coat 36 without lowering the surface temperature of the flexible tube member 40 when the flexible tube member 40 is heated. Therefore, a high fusing effect is achieved when adhering the outer coat 36 to the mesh tube 34 of the flexible tube member 40 by fusion bonding. The temperature for softening the outer coat 36 may be achieved only by heating the flexible tube member 40 only to a minimum required degree so that the outer coat 36 can be bonded on the outer periphery of the flexible tube member 40.

Then, when both ends of the core member 38 are pulled, the outer diameter of the core member 38 is reduced, and hence the outer peripheral surface of the core member 38 is separated from a state in which the outer peripheral surface is in close contact with the inner peripheral surface of the flex 32. In this state, the core member 38 is pulled out from the flex 32.

When the near infrared ray having a wavelength at the moment when the maximum value of emission spectrum in the range from 0.8 μm to 2.0 μm is used, the core member 38 of synthetic resin material of the flexible tube member 40 hardly absorbs heat (hardly heated). On the other hand, the mesh tube 34 formed of metallic material used on the surface of the flexible tube member 40 easily absorbs heat (easily heated). Such a light having the wavelength in the range of the near infrared ray is quite effective for core member 38, which is unwanted to be heated, disposed inside the mesh tube 34 or the flex 32. Therefore, the core member 38 can be maintained to a desirable shape or size when forming the flexible tube 26. In other words, when the mesh tube formed of metal is heated by the near infrared ray, the heated degree of the core member 38 is low, thereby causing little deformation in the core member 38. Consequently, change of the outer diameter of the core member 38 can be prevented while maintaining the cross-section of the core member 38 in a circular shape.

Hereinafter, effectiveness of usage of the near infrared ray having the wavelength, for example, in the range from 0.8 μm to 2.0 μm when the maximum value of emission spectrum is obtained immediately before coating the outer periphery of the flexible tube member 40 with the outer coat 36 will be clarified using some data.

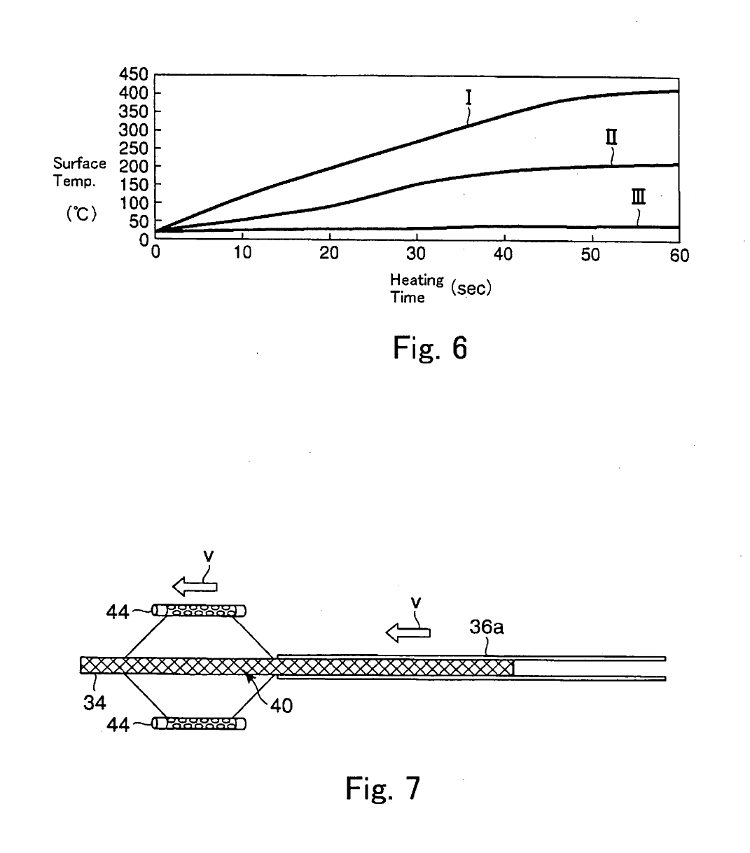

FIG. 4 shows heat absorption coefficients of metallic material (stainless steel is used here) and synthetic resin material (silicone rubber is used here) with respect to the wavelength of light emitted from the light-emitting member. FIG. 5 shows surface temperatures of round rods of 11 mm in outer diameter formed of metal and synthetic resin, respectively, to a light-emitting (heating time) when the light-emitting member is light-emitted with a prescribed output. Reference sign α designates the metallic material and reference sign β designates the synthetic resin material in FIG. 4 and FIG. 5. FIG. 6 shows actual surface temperatures of the flexible tube member 40 when the near infrared ray and infrared ray having a medium wavelength are irradiated on the flexible tube member 40 and when the flexible tube member 40 is placed in the atmosphere furnace at 450° C. with respect to the light emitting time (heating period). In FIG. 6, reference sign I designates a temperature-time behavior when the near infrared ray is irradiated to the flexible tube member 40, reference sign II designates the temperature-time behavior when the infrared ray is irradiated to the flexible tube member 40, and reference sign III designates the temperature-time behavior when the flexible tube member 40 is placed in the atmosphere furnace.

As shown in FIG. 4, whether or not the heat absorption coefficient of a subject varies depending on the difference of the wavelength of light emitted from the light-emitting member was studied. Metallic material (stainless steel) α and synthetic resin material (silicone rubber material) β formed into a sheet-shape were prepared as the subjects. The synthetic resin material β is the same as that used in the core member 38 of the flexible tube member 40. The metallic material α is the same as that used in the mesh tube 34 of the flexible tube member 40. In this case, the light-emitting members of the infrared heater 44 used here emit the same wavelength under the respective conditions.

As a result, it is clear that the levels of heat absorption coefficients of the metallic material α and the synthetic resin material β are counterchanged at a value in the range from 2.0 μm to 2.5 μm in wavelength. The heat absorption coefficient of the metallic material α is higher than the synthetic resin material β until the value of about 2.3 μm. In particular, in the range where the wavelength is from 0.8 μm to 2.0 μm, the heat absorption coefficient of the metallic material α is higher than twice the value of the synthetic resin material β, which can be said to be sufficiently high. Therefore, in the range of wavelength from 0.8 μm to 2.0 μm, the metallic material α maintains superiority to the synthetic resin material β in terms of heat absorption coefficient with respect to the light having the wavelength in the range of the near infrared ray.

Based on this result, a sample of the flexible tube member 40 was used to study the relation between the heating time and the surface temperature utilizing the light-emitting member of the infrared heater 44 that emits the aforementioned near infrared ray. The sample is formed to have a shape close to the flexible tube member 40, that is, a column shape having an outer diameter of about 11 mm which is almost the same as the outer diameter of the flexible tube 26.

As shown in FIG. 5, when comparing the time periods that are required to heat up the metallic material α and the synthetic resin material β from the room temperature to 120° C., which is an average softening temperature of the synthetic resin material β (an average temperature required for softening/melting the outer coat 36 formed of the aforementioned material), the metallic material α is heated up faster than the synthetic resin material β. It takes about one to two seconds for the metallic material α, and three seconds for the synthetic resin material β. Therefore, there is a difference of about twice in time. This is a result obtained under the condition in which the near infrared ray is not blocked by other members. In other words, it is the result obtained when the light from the light-emitting member of the infrared heater 44 is directly irradiated on the metallic material α and the synthetic material β without any blocking object.

In a state in which it is used for the core member 38 of the actual flexible tube member 40, since the core member 38 is covered by the metallic material α, such as the mesh tube 34 or the flex 32, the synthetic resin β needs longer time to be heated to the same temperature than the result shown in FIG. 5. In particular, since the flex 32 is disposed between the mesh tube 34 and the core member 38 in a movable state and not in an adhered state, heat transfer is prevented. Therefore, since the heat absorption coefficient of the synthetic resin material β is lower than the metallic material α, the core member 38 formed of the synthetic resin material β inserted into the flexible tube member 40 as the jig does not have enough time to be heated to a temperature that causes deformation such as expansion or melting only by heating the metallic material α to a required temperature. In other words, the core member 38 does not reach its deformation temperature, which could cause a problem when covering the outer coat 36. Therefore, even when the flex 32 and the mesh tube 34 are heated, for example, to 120° C., the core member 38 is maintained at a temperature which is too low to deform, and hence deformation such as expansion is prevented. In this manner, in view of such a result, it is recognized that the effectiveness of this technology employing the near infrared ray is significantly high.

As shown in FIG. 6, heating of the flexible tube member 40 can be described as follows. With the method of heating using the near infrared ray I, temperature increase with respect to time is faster than other methods, such as the case of using the infrared ray II of middle wavelength or the case of being placed in the atmosphere furnace III, and hence the surface temperature of the flexible tube member 40 can be increased to a desired temperature in a short time. Therefore, by using the near infrared ray I, heating time required for heating the surface temperature of the mesh tube 34 of the flexible tube member 40 to a desired temperature (120° C.) may be shortened in comparison with the case of using the infrared ray II. In this case, when the near infrared ray I is used, the temperature increases to the desired temperature (120° C.) in about eight to nine seconds, while the infrared ray II requires about twenty-three to twenty-four seconds to raise the temperature of the flexible tube member to the desired temperature (120° C.). When the flexible tube member 40 is placed in the atmosphere furnace III, it took about 30 minutes to rise the surface temperature of the mesh tube 34 to the desired surface temperature (120° C.). Therefore, the time required for manufacturing the flexible tube 26 can be shortened by using the light-emitting member that emits the near infrared ray in the infrared heater 44.

As described above, according to the present embodiment, the following effects are achieved.

By using the light-emitting member that emits light with wavelength in the range of near infrared ray, the surface of the flexible tube member 40 (mesh tube 34) of metallic material can be heated efficiently within a short time to rise the temperature of the outer coat 36 to a temperature required to cause the outer coat 36 to get into the clearances on the mesh tube 34, and the temperature of the core member 38 of synthetic resin material, heating of which is not desired, can be prevented from increasing. Therefore, the core member 38 can prevent occurrence of deformation such as expansion, and hence deterioration of appearance of the surface of the outer coat 36 due to deformation of the core member 38 or unevenness of a bonding force between the outer coat 36 and the flexible tube member 40 can be prevented. Therefore, the flexible tube 26 in which the outer peripheral surface of the mesh tube 34 of the flexible tube member 40 and the inner peripheral surface of the outer coat 36 are bonded with a strong force is provided.

When pulling the core member 38 from inside the flex 32, the outer diameter of the core member 38 can be reduced. Therefore, generation of friction between the flex 32 and the core member 38 can be prevented. Accordingly, even when the core member 38 is pulled out from inside the flex 32, the flex 32 can be maintained in its predetermined helical shape, and the helical shape can be prevented from deforming.

Since the heating time for covering the outer coat 36 on the flexible tube member 40 can be significantly reduced in comparison with the case in which the infrared ray of the middle wavelength is used, the time required for manufacturing may be reduced as well.

Data shown in FIG. 4 to FIG. 6 are results obtained when the material is selected as discussed above, and may be varied according to the materials chosen for any particular application. Therefore, the wavelength when the maximum value of emission spectrum of the near infrared ray, which is emitted from the respective light emitting members of the infrared heater 44, is obtained is not limited to the range from 0.8 μm to 2.0 μm, and for example, by changing the materials of the mesh tube 34, the outer coat 36 and the core member 38, the wavelength can be varied as needed within the range of the near infrared ray.

Subsequently, referring to FIG. 7, a second embodiment will be described. This embodiment is a modification of the first embodiment, and the same parts as described in the first embodiment will be represented by the same reference numerals.

In this embodiment, when the outer periphery of the flexible tube member 40 is covered with the outer coat 36, the flexible tube 26 is manufactured by covering an outer coat 36a which is formed into tubular shape in advance on the outside of the flexible tube 26 instead of extrusion molding or dipping molding.

As shown in FIG. 7, the flexible tube member 40 is heated by the infrared heater 44. The light-emitting member that emits light in the range of the near infrared ray of the infrared heater 44 is caused to emit light and irradiate the light on the outer surface of the flexible tube member 40 entirely and evenly to heat the flexible tube member 40. At this time, the inner peripheral surface of the outer coat 36a is heated to a temperature that can make the outer coat 36a possible to impregnate into the mesh tube 34.

Immediately after this, the outer peripheral surface of the flexible tube member 40 is covered with the tubular outer coat 36a. At this time, the flexible tube member 40 is heated while moving the infrared heater 44, that is, the light-emitting member at a velocity v, which is the same velocity as the velocity v to cover the outer coat 36a on the flexible tube member 40 from the right to the left in FIG. 7, simultaneously with the movement to cover the tubular outer coat 36a formed in advance on the outer periphery of the flexible tube member 40. In other words, the outer coat 36a and the infrared heater 44 are moved in the same direction at the same velocity v while maintaining the flexible tube member 40 stationary with respect to the outer coat 36a and infrared heater 44. Therefore, the flexible tube member 40 heated to the softening temperature of the outer coat 36a is covered with the outer coat 36a. Accordingly, the outer peripheral surface of the mesh tube 34 of the flexible tube member 40 and the inner peripheral surface of the outer coat 36a are fused and bonded.

In this embodiment, the operation that the outer coat 36a and the infrared heater 44 are moved with respect to the flexible tube member 40 in the same direction at the same velocity v has been described. As a matter of course, it is also possible to move the flexible tube member 40 in a state in which the outer coat 36a and the infrared heater 44 are retained at a predetermined position to bond the outer coat 36a and the flexible tube member 40.

According to this embodiment, the same effect that can be achieved in the first embodiment is achieved.

The flexible tube 26 of the insertion portion 12 of the endoscope 10 has been described in the first and the second embodiment. However, it can also be applied also when it is used for the universal cord 16. Also, the flexible tube 26 that is used for the endoscope 10 for medical use has been described here, it can also be applied to the flexible tube for the endoscope for industrial use.

Several embodiments have been described so far in detail referring to the drawings, the present invention is not limited to the above-described embodiments, and includes all the implementations performed without departing from the scope of the invention.

According to the description above, the invention as stated in the following terms is achieved. Also, a combination of the respective terms is possible.

While there has been shown and described what is considered to be preferred embodiments of the invention, it will, of course, be understood that various modifications and changes in form or detail could readily be made without departing from the spirit of the invention. It is therefore intended that the invention be not limited to the exact forms described and illustrated, but should be constructed to cover all modifications that may fall within the scope of the appended claims.

1. A method of manufacturing an endoscope flexible tube comprising:
irradiating a flexible tube member with a near infrared ray before covering the flexible tube member formed at least partly of metal with an outer coat to raise the surface temperature of the flexible tube member to a temperature higher than that the outer coat deforms; and
covering an outer periphery of the flexible tube member raised in temperature by the near infrared ray with the outer coat.

2. A method of manufacturing an endoscope flexible tube according to claim 1, wherein the method of manufacturing the flexible tube member comprises:
winding a flex on a cylindrical or column-shaped core member formed of material containing at least synthetic resin; and
disposing a mesh tube containing metal as at least part of the material on the outer periphery of the flex.

3. A method of manufacturing an endoscope flexible tube according to claim 2, further comprising:
decreasing an outer diameter of the core member and pulling the core member from the flexible tube member after covering the outer periphery of the flexible tube member with the outer coat.

4. A method of manufacturing an endoscope flexible tube according to claim 1, wherein a wavelength when a maximum value of emission spectrum of the near infrared ray is obtained resides within the range from about 0.8 μm to about 2.0 μm.

5. A method of manufacturing an endoscope flexible tube according to claim 1, wherein the covering of the outer periphery of the flexible tube member is performed by one of extrusion molding and dipping.

6. A method of manufacturing an endoscope flexible tube according to claim 1, wherein the covering of the outer periphery of the flexible tubular member with the outer coat comprises molding the outer coat into a tubular shape in advance of the covering.

7. A method of manufacturing an endoscope flexible tube according to claim 1, wherein the endoscope flexible tube is an insertion portion of an endoscope.

8. A method of manufacturing an endoscope flexible tube according to claim 1, wherein the endoscope flexible tube is a universal cord of an endoscope.

9. A method of manufacturing an endoscope flexible tube by covering an outer periphery of a mesh tube whereof at least an element wire or a part of a bundle of element wires is formed of metallic material with an outer coat formed of a thermoplastic resilient member by extrusion molding or dipping, the method comprising: heating a surface of the mesh tube using a light emitting member for emitting a near infrared ray whereof the maximum value of emission spectrum resides within the range from about 0.8 μm to about 2.0 μm in advance before covering the outer periphery of the mesh tube with the outer coat; and bonding between the mesh tube and the outer coat by an energy generated when preheating the mesh tube.

10. A method of manufacturing an endoscope flexible tube by covering an outer periphery of a mesh tube whereof at least an element wire or a part of a bundle of element wires is formed of metallic material with an outer coat formed of a thermoplastic resilient member and formed into a tubular shape in advance, the method comprising: heating a surface of the mesh tube using a light emitting member of a near infrared ray whereof the maximum value of emission spectrum resides in the range from about 0.8 μm to about 2.0 μm before covering the outer periphery of the mesh tube with the thermoplastic resilient member; and bonding the mesh tube and the thermoplastic resilient member with an energy generated when preheating the mesh tube.

11. A method of manufacturing an endoscope flexible tube comprising:
disposing a mesh tube comprising an element wire or a bundle of element wires formed at least partly of metallic material weaved therein outside a flex which is a metal band strip wound into a helical shape;
irradiating a near infrared ray from outside the mesh tube to heat the mesh tube to a temperature at which an outer coat formed of thermoplastic resilient member for covering the outside of the mesh tube is at least softened; and
after having heated the mesh tube to the temperature at which the outer coat is softened, covering the outer periphery of the mesh tube with the outer coat by one of extrusion molding and dipping to bond the mesh tube and the outer coat by preheating of the mesh tube.

12. A method of manufacturing an endoscope flexible tube according to claim 11, wherein the wavelength of the near infrared ray irradiated in the step of heating resides within the range from about 0.8 μm to about 2.0 μm.

13. A method of manufacturing an endoscope flexible tube comprising:
detachably disposing a flex formed by winding a band strip into a helical shape on an outside of a core member, the core member having a circumferential peripheral surface and being capable of expanding and contracting in a radial direction and a longitudinal direction;
disposing a mesh tube on an outside of the flex, the mesh tube including an element wire or a bundle of element wires formed at least partly of metallic material weaved therein and having a higher heat absorption coefficient observed when a near infrared ray is irradiated than the core member;
irradiating the near infrared ray from outside the mesh tube and heating the mesh tube to a temperature at which an outer coat formed of thermoplastic resilient member for covering the mesh tube is softened;
covering an outer periphery of the mesh tube with the outer coat by one of extrusion molding and dipping immediately after having heated the mesh tube to the temperature at which the outer coat is softened and bonding the mesh tube and the outer coat by preheating the mesh tube; and
removing the core member from inside the mesh tube in a state in which the core member is pulled in the longitudinal direction to reduce the diameter radially inwardly.

14. A method of manufacturing an endoscope flexible tube according to claim 13, wherein stainless steel is used for the mesh tube,
silicone rubber is used for the core member, and
light whereof the wavelength of which can obtain the maximum value of emission spectrum resides within the range from about 0.8 μm to about 2.0 μm is irradiated as the near infrared ray.

15. A method of manufacturing an endoscope flexible tube comprising:
detachably disposing a flex formed by winding a band strip into helical shape on an outside of a core member, the core member having a circumferential peripheral surface and being capable of expanding and contracting in a radial direction and a longitudinal direction;
disposing a mesh tube on an outside of the flex, the mesh tube including an element wire or a bundle of element wires formed at least partly of metallic material weaved therein and having a higher heat absorption coefficient observed when a near infrared ray is irradiated than the core member;
irradiating the near infrared ray from outside the mesh tube and heating the mesh tube to a temperature at which an outer coat formed of thermoplastic material of tubular shape for covering the mesh tube;
covering an outer periphery of the mesh tube with the outer coat immediately after having heated the mesh tube to the temperature at which the outer coat is softened and bonding the mesh tube and the outer coat by preheating the mesh tube; and
removing the core member from inside the mesh tube in a state in which the core member is pulled in the longitudinal direction to reduce the diameter radially inwardly.

16. A method of manufacturing an endoscope flexible tube according to claim 15, wherein stainless steel is used for the mesh tube;
silicone rubber is used for the core member; and
light whereof the wavelength of which can obtain the maximum value of emission spectrum resides within the range from about 0.8 μm to about 2.0 μm is irradiated as the near infrared ray.

17. An endoscope flexible tube manufactured by a method comprising: detachably disposing a flex formed by winding a band strip into a helical shape on an outside of a core member, the core member having a circumferential peripheral surface and being capable of expanding and contracting in a radial direction and a longitudinal direction;
disposing a mesh tube on an outside of the flex, the mesh tube including an element wire or a bundle of element wires formed at least partly of metallic material weaved therein and having a higher heat absorption coefficient with respect to a near infrared ray than the core member when a surface of the mesh tube is heated by the near infrared ray;
heating an outer periphery of the mesh tube by the near infrared ray to a temperature at which an outer coat of thermoplastic resilient member for covering the outer periphery of the mesh tube is at least softened and bonded to the mesh tube;
immediately after the heating, covering the outer peripheral surface of the mesh tube with the outer coat by one of extrusion molding and dipping and bonding the mesh tube and the outer coat by preheating the mesh tube; and
pulling the core member out from the flex in a state in which the core member is pulled in the longitudinal direction to reduce the diameter radially inwardly.

18. An endoscope flexible tube according to claim 17, wherein the mesh tube is formed of metallic material containing at least one of stainless steel alloy, copper, brass, tungsten, and iron, and the core member is formed of a synthetic resin material containing silicone rubber.

19. An endoscope flexible tube according to claim 17, wherein the mesh tube is formed of a compound of metallic material containing at least one of stainless steel alloy, copper, brass, tungsten and iron and non-metallic material containing at least one of synthetic resin, silk string, and kite string, and the core member is formed of a synthetic resin material containing silicone rubber material.

20. An endoscope flexible tube according to claim 17, wherein the mesh tube is formed of stainless steel,
the core member is formed of silicone rubber, and
a wavelength whereby the maximum value of emission spectrum of the near infrared ray can be obtained resides in the range from about 0.8 μm to about 2.0 μm.

21. An endoscope flexible tube manufactured by a method comprising: detachably disposing a flex formed by winding a band strip into a helical shape on an outside of a core member, the core member having a circumferential peripheral surface and being capable of expanding and contracting in a radial direction and a longitudinal direction;
disposing a mesh tube on an outside of the flex, the mesh tube including an element wire or a bundle of element wires formed at least partly of metallic material weaved therein and having higher a heat absorption coefficient with respect to a near infrared ray than the core member when a surface of the mesh tube is heated by the near infrared ray;
heating an outer periphery of the mesh tube by the near infrared ray to a temperature at which an outer coat formed of a thermoplastic resilient material for covering the outer periphery of the mesh tube into a tubular shape is at least softened and bonded to the mesh tube;
immediately after the heating, covering an outer peripheral surface of the mesh tube and bonding the mesh tube and the outer coat by preheating the mesh tube; and
pulling the core member out from the flex in a state in which the core member is pulled in the longitudinal direction to reduce the diameter radially inwardly.

22. An endoscope flexible tube according to claim 21, wherein the mesh tube is formed of metallic material containing at least one of stainless steel alloy, copper, brass, tungsten, and iron; and
the core member is formed of a synthetic resin material containing silicone rubber.

23. An endoscope flexible tube according to claim 21 wherein the mesh tube is formed of a compound including metallic material containing at least one of stainless steel alloy, copper, brass, tungsten, and iron and non-metallic material containing at least one of a synthetic resin, silk string, and kite string; and
the core member is formed of a synthetic resin material containing silicone rubber.

24. An endoscope flexible tube according to claim 21, wherein the mesh tube is formed of stainless steel,
the core member is formed of silicone rubber, and
a wavelength whereby the maximum value of emission spectrum of the near infrared ray can be obtained resides within the range from about 0.8 μm to about 2.0 μm.
